(19) United States
(12) Patent Application Publication
(10) Pub. No.: US 2008/0138298 A1
(43) Pub. Date: Jun. 12, 2008

(54) Oral care compositions comprising zinc and phytate

(75) Inventors: William Michael Glandorf, Mason, OH (US); Xiaoli Wang, Beijing (CN); James Robert Schwartz, West Chester, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/637,590

(22) Filed: Dec. 12, 2006

(51) Int. Cl. A61K 8/55; A61K 8/69; A61Q 11/00

(52) U.S. Cl. 424/52; 424/57

(57) ABSTRACT

Disclosed are oral care compositions and their use, comprising in an orally acceptable carrier:

(a) from about 0.01 to about 10% by weight of an essentially water-insoluble zinc compound, and

(b) from about 0.01% to about 10% by weight of a compound having C—O—P bonds selected from polyphosphorylated inositol compounds such as phytic acid, myo-inositol pentakis(dihydrogen phosphate); myo-inositol tetrakis(dihydrogen phosphate), myo-inositol trikis(dihydrogen phosphate), and an alkali metal, alkaline earth metal or ammonium salt thereof.

The compositions are effective in preventing and controlling oral cavity conditions including plaque, calculus, caries, periodontal disease, mouth malodor and dental erosion and have acceptable aesthetics without the unpleasant astringent and metallic taste associated with the use of zinc.

### FIELD OF THE INVENTION

The present invention relates to oral care compositions comprising an essentially water-insoluble zinc compound and a phytate compound.

### BACKGROUND OF THE INVENTION

The use of zinc compounds in oral care products such as mouthwashes, rinses and toothpastes is a widely accepted practice. Zinc has been used for its ability to neutralize oral malodor and to provide antimicrobial, antiplaque and anticalculus activities. The activity of zinc compounds is generally attributed to zinc ions, in particular divalent zinc ions (Zn⁺²). Thus, water-soluble and highly ionized zinc compounds such as zinc chloride that readily provide active zinc ions have found utility in oral compositions, which are typically aqueous based. However, the soluble zinc compounds have the disadvantages of leaving an unpleasant astringent and metallic taste in the mouth as well as having short-lived efficacy against plaque, calculus and as an odor inhibitor. Sparingly water-soluble salts such as zinc citrate and zinc lactate have thus been used to moderate the release of zinc ions, thereby reducing astringency and providing slow dissolution of the zinc compound by saliva for longer activity in the oral cavity. The sparingly soluble characteristic of these zinc compounds promotes longevity of action at the expense of initial or immediate efficacy. The use of zinc compounds of varying solubility has been disclosed for example, in U.S. Pat. Nos. 4,082,841; 4,100,269; 4,022,880; 4,138,477; 4,144,323; 4,154,815; 4,289,755; 4,325,939; 4,339,432; 4,425,325; 4,416,867; 4,469,674; 4,522,806; 4,568,540; 4,647,452; 4,664,906; 4,814,163; 4,814,164; 4,992,259; 5,000,944; 5,085,850; 5,188,820; 5,455,024; 5,456,902; 5,587,147; 5,855,873; 6,015,547; 6592,849; 6,723,305.

The use of phytic acid and phytate salts in oral care products has also been the subject of previous disclosures, focusing on the anticaries, anticalculus, chelant, and anti-staining activities of these compounds, such as described in U.S. Pat. Nos. 4,259,316; 4,335,102; 4,305,928; 4,394,371; 4,528,181; 4,826,675; 5,281,410; 5,286,479; 5,300,289; 5,762,911; and 5,891,448; in WO 02/02060; WO 04/024112; WO 04/045594; JP04036229A2; JP10087458A2; JP10182383A2; JP11021216A2; JP11171749A2; JP11349460A2; JP56018911A2; JP56018912A2; JP56018913A2; JP56022721A2; JP56039008A2; JP56045408A2; JP56075422A2; JP2001233750A2; and JP2003335646A2.

While both zinc compounds and phytate compounds have been recommended for various purposes in oral compositions, there is no suggestion in any of the known art that the combination of an insoluble zinc compound and a phytate compound in an oral care composition would be particularly effective in preventing and controlling oral cavity conditions including calculus, plaque, caries, periodontal disease and mouth malodor. The present compositions take advantage of such combination and importantly provide long-lasting effects while avoiding the undesirable astringent and metallic taste associated with the use of zinc.

### SUMMARY OF THE INVENTION

The present invention is directed to oral care compositions and their use, comprising in an orally acceptable carrier:

(a) from about 0.01 to about 10% by weight of an essentially water-insoluble zinc compound, and

(b) from about 0.01% to about 10% by weight of a compound having C—O—P bonds selected from polyphosphorylated inositol compounds such as phytic acid, myo-inositol pentakis(dihydrogen phosphate); myo-inositol tetrakis(dihydrogen phosphate), myo-inositol trikis(dihydrogen phosphate), and an alkali metal, alkaline earth metal or ammonium salt thereof.

The compositions are effective in preventing and controlling oral cavity conditions including plaque, calculus, caries, periodontal disease and mouth malodor and have acceptable aesthetics without the unpleasant astringent and metallic taste associated with the use of zinc.

These and other features, aspects, and advantages of the invention will become evident to those skilled in the art from a reading of the present disclosure.

### DETAILED DESCRIPTION OF THE INVENTION

While the specification concludes with claims particularly pointing out and distinctly claiming the invention, it is believed that the present invention will be better understood from the following description.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

Herein, “comprising” means that other steps and other components which do not affect the end result can be added. This term encompasses the terms “consisting of” and “consisting essentially of.”

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

By “oral care composition” or “oral composition” is meant a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. In addition to cleaning teeth to remove dental plaque, oral care compositions function to prevent the formation of dental calculus and disorders such as caries, periodontitis and gingivitis, and also to eliminate and prevent oral malodor or halitosis and staining. Examples of oral care product forms include toothpaste, dentifrice, tooth gel, subgingival gel, mouthrinse, mouthspray, mousse, foam, denture product, lozenge, chewable tablet or chewing gum and strips or films for direct application or attachment to oral surfaces.

The term “dentifrice”, as used herein, means paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having the gel surrounding the paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices.

The term “teeth”, as used herein, refers to natural teeth as well as artificial teeth or dental prosthesis.

Herein, the terms “tartar” and “calculus” are used interchangeably and refer to mineralized dental plaque biofilms.

The term “orally acceptable carrier” as used herein includes any safe and effective materials for use in the compositions of the present invention. Such materials include conventional additives in oral care compositions including but not limited to fluoride ion sources, anti-calculus or anti-tartar agents, desensitizing agents, teeth whitening agents such as peroxide sources, abrasives such as silica, herbal agents, chelating agents, buffers, anti-staining agents, alkali metal bicarbonate salts, thickening materials, humectants, water, surfactants, titanium dioxide, flavor system, sweetening agents, xylitol, coloring agents, and mixtures thereof.

Active and other ingredients useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

The present oral care compositions comprise as essential ingredients an essentially water-insoluble zinc compound and a complexing agent selected from polyphosphorylated inositol compounds such as phytic acid, myo-inositol pentakis(dihydrogen phosphate); myo-inositol tetrakis(dihydrogen phosphate), myo-inositol trikis(dihydrogen phosphate), and an alkali metal or ammonium salt thereof. Phytic acid, also known as myo-inositol 1,2,3,4,5,6-hexakis (dihydrogen phosphate) or inositol hexaphosphoric acid, and its alkali metal, alkaline earth metal or ammonium salts are the preferred compounds containing C—O—P bonds, functioning as complexing agent to aid in solubilizing the essentially water-insoluble zinc compound to provide a supply of divalent zinc ions (Zn⁺²), which function as antimicrobial, antiplaque, anticalculus and deodorizing agent. Herein, the term “phytate” includes phytic acid and its salts as well as the other polyphosphorylated inositol compounds. Phytates also act as anticaries agent and as inhibitor of hydroxyapatite or calculus formation.

The term “essentially water-insoluble” as employed herein in reference to zinc compounds, means that the zinc-containing compound has a solubility in water that is less than about 0.1 gram per 100 milliliters of water at 25° C. Examples of essentially water insoluble zinc compounds useful herein include zinc carbonate, zinc oxide, zinc silicate, zinc phosphate, zinc pyrophosphate, and zinc-containing minerals such as smithsonite, hydrozincite (zinc carbonate hydroxide), aurichalcite and rosasite. A preferred zinc compound is zinc carbonate, which term as used herein includes various forms including a crystalline form also referred to as basic zinc carbonate, which is commercially available as Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice.

In accordance with certain aspects of the present invention, oral care compositions are provided comprising in an orally acceptable carrier, from about 0.01 to about 10% by weight of a phytate compound and from about 0.01% to about 10% by weight of one or a mixture of essentially insoluble zinc compounds. In a number of embodiments, the essentially insoluble zinc compound is zinc carbonate, zinc oxide or zinc pyrophosphate and the phytate compound is phytic acid or its alkali metal or ammonium salt. The level of insoluble zinc compound in these embodiments is up to about 10%, typically from about 0.01% to about 5%. The level of phytate compound is up to about 10%, typically from about 0.01% to about 5%.

While it is believed that the divalent zinc ions generally possess the activities beneficial for oral cavity treatment, an important consideration in this respect is that the final product must not be so excessively astringent or unpleasant tasting as to be unacceptable to the user. Many soluble or sparingly soluble zinc compounds including zinc chloride, zinc acetate, zinc sulfate and zinc citrate that readily provide active zinc ions are known to be highly astringent when incorporated in aqueous oral compositions. Thus the present invention utilizes essentially insoluble zinc compounds which tend to be significantly less astringent than the soluble or sparingly-soluble zinc compounds. The phytate compounds are present in the compositions to provide a solubilizing function by complexing with zinc and providing a source of active divalent zinc ions. It is also believed that the zinc/phytate complex and some of the insoluble zinc compounds deposit on teeth and other oral surfaces, thereby providing a reservoir of zinc ions released over a prolonged period of time. Soluble zinc salts normally would simply be washed away with water during rinsing or with saliva, and thus may not provide long lasting activity.

The oral care composition of the present invention may be in various forms including toothpaste, dentifrice, tooth gel, subgingival gel, mouthrinse, mouthspray, mousse, foam, denture product, lozenge, chewable tablet or chewing gum. The oral care composition may also be incorporated onto strips or films for direct application or attachment to oral surfaces.

The present compositions will optimally have a pH ranging from about 4.0 to about 10.0. In a number of embodiments, the pH of the compositions is from about 6.0 to about 9.0. The pH of a dentifrice composition is measured from a 3:1 aqueous slurry of the dentifrice, e.g., 3 parts water to 1 part toothpaste.

In addition to the components described above, the present compositions may comprise additional optional components collectively referred to as orally acceptable carrier materials, which are described in the following paragraphs.

### Orally Acceptable Carrier Materials

The orally acceptable carrier comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy.

The carriers or excipients of the present invention can include the usual and conventional components of dentifrices, non-abrasive gels, subgingival gels, mouthwashes or rinses, mouth sprays, chewing gums, lozenges and breath mints as more fully described hereinafter.

The choice of a carrier to be used is basically determined by the way the composition is to be introduced into the oral cavity. Carrier materials for toothpaste, tooth gel or the like include abrasive materials, sudsing agents, binders, humectants, flavoring and sweetening agents, etc. as disclosed in e.g., U.S. Pat. No. 3,988,433 to Benedict. Carrier materials for biphasic dentifrice formulations are disclosed in U.S. Pat. No. 5,213,790 issued May 23, 1993; U.S. Pat. No. 5,145,666 issued Sep. 8, 1992; and U.S. Pat. No. 5,281,410 issued Jan. 25, 1994 all to Lukacovic et al. and in U.S. Pat. Nos. 4,849,213 and 4,528,180 to Schaeffer. Mouthwash, rinse or mouth spray carrier materials typically include water, flavoring and sweetening agents, etc., as disclosed in, e.g., U.S. Pat. No. 3,988,433 to Benedict. Lozenge carrier materials typically include a candy base; chewing gum carrier materials include a gum base, flavoring and sweetening agents, as in, e.g., U.S. Pat. No. 4,083,955 to Grabenstetter et al. Sachet carrier materials typically include a sachet bag, flavoring and sweetening agents. For subgingival gels used for delivery of actives into the periodontal pockets or around the periodontal pockets, a “subgingival gel carrier” is chosen as disclosed in, e.g. U.S. Pat. Nos. 5,198,220 and 5,242,910 issued Mar. 30, 1993 and Sep. 7, 1993, respectively both to Damani. Carriers suitable for the preparation of compositions of the present invention are well known in the art. Their selection will depend on secondary considerations like taste, cost, and shelf stability, etc.

The compositions of the present invention may be in the form of non-abrasive gels and subgingival gels, which may be aqueous or non-aqueous. Aqueous gels generally include a thickening agent (from about 0.1% to about 20%), a humectant (from about 10% to about 55%), a flavoring agent (from about 0.04% to about 2%), a sweetening agent (from about 0.1% to about 3%), a coloring agent (from about 0.01% to about 0.5%), and the balance water. The compositions may comprise an anticaries agent (from about 0.05% to about 0.3% as fluoride ion), and an anticalculus agent (from about 0.1% to about 13%).

In one embodiment, the compositions of the subject invention are in the form of dentifrices, such as toothpastes, tooth gels and tooth powders. Components of such toothpaste and tooth gels generally include one or more of a dental abrasive (from about 6% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion) and an anticalculus agent (from about 0.1% to about 13%). Tooth powders, of course, contain substantially all non-liquid components.

Other embodiments of the subject invention are mouthwashes or rinses and mouth sprays. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion) and an anticalculus agent (from about 0.1% to about 3%).

The compositions of the subject invention may also be in the form of dental solutions and irrigation fluids. Components of such dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

Chewing gum compositions typically include one or more of a gum base (from about 50% to about 99%), a flavoring agent (from about 0.4% to about 2%) and a sweetening agent (from about 0.01% to about 20%).

The term “lozenge” as used herein includes: breath mints, troches, pastilles, microcapsules, and fast-dissolving solid forms including freeze dried forms (cakes, wafers, thin films, tablets) and compressed tablets. The term “fast-dissolving solid form” as used herein means that the solid dosage form dissolves in less than about 60 seconds, preferably less than about 15 seconds, more preferably less than about 5 seconds, after placing the solid dosage form in the oral cavity. Fast-dissolving solid forms are disclosed in commonly-assigned WO 95/33446 and WO 95/11671; U.S. Pat. No. 4,642,903; U.S. Pat. No. 4,946,684; U.S. Pat. No. 4,305,502; U.S. Pat. No. 4,371,516; U.S. Pat. No. 5,188,825; U.S. Pat. No. 5,215,756; U.S. Pat. No. 5,298,261; and U.S. Pat. No. 4,687,662.

Lozenges include discoid-shaped solids comprising a therapeutic agent in a flavored base. The base may be a hard sugar candy, glycerinated gelatin or combination of sugar with sufficient mucilage to give it form. These dosage forms are generally described in Remington: The Science and Practice of Pharmacy, 19^{th} Ed., Vol. II, Chapter 92, 1995. Lozenge compositions (compressed tablet type) typically include one or more fillers (compressible sugar), flavoring agents, and lubricants. Microcapsules of the type contemplated herein are disclosed in U.S. Pat. No. 5,370,864 to Peterson et al., issued Dec. 6, 1994.

In still another aspect, the invention provides a dental implement impregnated with the present composition. The dental implement comprises an implement for contact with teeth and other tissues in the oral cavity, said implement being impregnated with the present composition. The dental implement can be impregnated fibers including dental floss or tape, chips, strips, films and polymer fibers.

Types of orally acceptable carriers or excipients which may be included in compositions of the present invention, along with specific non-limiting examples, are discussed in the following paragraphs.

### Fluoride Source

It is common to have a water-soluble fluoride compound present in dentifrices and other oral compositions in an amount sufficient to give a fluoride ion concentration in the composition, and/or when it is used of from about 0.0025% to about 5.0% by weight, preferably from about 0.005% to about 2.0% by weight, to provide anticaries effectiveness. A wide variety of fluoride ion-yielding materials can be employed as sources of soluble fluoride in the present compositions. Examples of suitable fluoride ion-yielding materials are found in U.S. Pat. No. 3,535,421, Oct. 20, 1970 to Briner et al. and U.S. Pat. No. 3,678,154, Jul. 18, 1972 to Widder et al. Representative fluoride ion sources include: stannous fluoride, sodium fluoride, potassium fluoride, sodium monofluorophosphate, indium fluoride, amine fluoride and many others. Stannous fluoride and sodium fluoride are preferred, as well as mixtures thereof.

### Abrasives

Dental abrasives useful in the topical, oral carriers of the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 issued to Cooley & Grabenstetter on Dec. 25, 1962. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. Examples include the silica xerogels marketed under the trade name “Syloid” by the W.R. Grace & Company, Davison Chemical Division and precipitated silica materials such as those marketed by the J. M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present invention are described in more detail in Wason, U.S. Pat. No. 4,340,583, issued Jul. 29, 1982; and in commonly-assigned U.S. Pat. No. 5,603,920, issued on Feb. 18, 1997; U.S. Pat. No. 5,589,160, issued Dec. 31, 1996; U.S. Pat. No. 5,658,553, issued Aug. 19, 1997; U.S. Pat. No. 5,651,958, issued Jul. 29, 1997, and U.S. Pat. No. 6,740,311, issued May 25, 2004.

Mixtures of abrasives can be used such as mixtures of the various grades of Zeodent® silica abrasives listed above. The total amount of abrasive in dentifrice compositions of the subject invention typically range from about 6% to about 70% by weight; toothpastes preferably contain from about 10% to about 50% of abrasives, by weight of the composition. Dental solution, mouth spray, mouthwash and non-abrasive gel compositions of the subject invention typically contain little or no abrasive.

### Anticalculus Agent

The present compositions may optionally include an additional anticalculus agent, such as a pyrophosphate salt as a source of pyrophosphate ion. The pyrophosphate salts useful in the present compositions include the dialkali metal pyrophosphate salts, tetraalkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, from about 1.5% to about 10% in one embodiment, and from about 2% to about 6% in another embodiment. Free pyrophosphate ions may be present in a variety of protonated states depending on the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is the preferred pyrophosphate salt in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, generally from about 1.5% to about 15%, preferably from about 2% to about 10%, and most preferably from about 3% to about 8%, by weight of the dentifrice composition.

Compositions may also comprise a mixture of dissolved and undissolved pyrophosphate salts. Any of the above mentioned pyrophosphate salts may be used.

The pyrophosphate salts are described in more detail in Kirk-Othmer Encyclopedia of Chemical Technology, 3rd Ed., Vol. 17, Wiley-Interscience (1982).

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., as well as, e.g., polyamino propane sulfonic acid (AMPS), polyphosphates (e.g., tripolyphosphate and hexametaphosphate), diphosphonates (e.g., EHDP; AFHP), polypeptides (such as polyaspartic and polyglutamic acids), and mixtures thereof.

Examples of phosphonate copolymers include the diphosphonate-derivatized polymers in U.S. Pat. No. 5,011,913 to Benedict et al, such as diphosphonate modified polyacrylic acid. Other suitable phosphonate-containing polymers are described in U.S. Pat. No. 5,980,776 to Zakikhani, et al. and U.S. Pat. No. 6,071,434 to Davis et al.

Polyphosphates may also be included in the present compositions. A polyphosphate is generally understood to consist of two or more phosphate groups arranged primarily in a linear configuration, although some cyclic derivatives may be present. In addition to pyrophosphates and tripolyphosphate, which are technically polyphosphates, also desired are the polyphosphates having an average of about four or more phosphate groups, i.e., tetrapolyphosphate and hexametaphosphate, among others. Polyphosphates larger than tetrapolyphosphate usually occur as amorphous glassy materials, the linear “glassy” polyphosphates having the formula:

XO(XPO₃)ₙX

wherein X is sodium or potassium and n averages from about 6 to about 125. Preferred polyphosphates are manufactured by FMC Corporation which are commercially known as Sodaphos (n≈6), Hexaphos (n≈13), and Glass H (n≈21). These polyphosphates may be used alone or in combination thereof.

### Chelating Agents

Another optional agent is a chelating agent, also called sequestrants, such as gluconic acid, tartaric acid, citric acid and pharmaceutically-acceptable salts thereof. Chelating agents are able to complex calcium found in the cell walls of the bacteria. Chelating agents can also disrupt plaque by removing calcium from the calcium bridges which help hold this biomass intact. However, it is not desired to use a chelating agent which has an affinity for calcium that is too high, as this may result in tooth demineralization, which is contrary to the objects and intentions of the present invention. Suitable chelating agents will generally have a calcium binding constant of about 10¹ to 10⁵ to provide improved cleaning with reduced plaque and calculus formation. Chelating agents also have the ability to complex with metallic ions and thus aid in preventing their adverse effects on the stability or appearance of products. Chelation of ions, such as iron or copper, helps retard oxidative deterioration of finished products.

Examples of suitable chelating agents are sodium or potassium gluconate and citrate; citric acid/alkali metal citrate combination; disodium tartrate; dipotassium tartrate; sodium potassium tartrate; sodium hydrogen tartrate; potassium hydrogen tartrate; sodium, potassium or ammonium polyphosphates and mixtures thereof. The amounts of chelating agent suitable for use in the present invention are about 0.1% to about 2.5%, preferably from about 0.5% to about 2.5% and more preferably from about 1.0% to about 2.5%.

Still other chelating agents suitable for use in the present invention are the anionic polymeric polycarboxylates. Such materials are well known in the art, being employed in the form of their free acids or partially or preferably fully neutralized water soluble alkali metal (e.g. potassium and preferably sodium) or ammonium salts. Examples are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymeric polycarboxylates include the 1:1 copolymers of maleic anhydride with ethyl acrylate, hydroxyethyl methacrylate, N-vinyl-2-pyrrolidone; or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Additional operative polymeric polycarboxylates are disclosed in U.S. Pat. No. 4,138,477, Feb. 6, 1979 to Gaffar and U.S. Pat. No. 4,183,914, Jan. 15, 1980 to Gaffar et al. and include copolymers of maleic anhydride with styrene, isobutylene or ethyl vinyl ether; polyacrylic, polyitaconic and polymaleic acids; and sulfoacrylic oligomers of M.W. as low as 1,000 available as Uniroyal ND-2.

### Teeth Whitening Actives

Teeth whitening actives may be included in the oral care compositions of the present invention. The actives suitable for whitening include the peroxides, metal chlorites, perborates, percarbonates, peroxyacids, persulfates, and combinations thereof. Suitable peroxide compounds include hydrogen peroxide, urea peroxide, calcium peroxide, and mixtures thereof. Suitable metal chlorites include calcium chlorite, barium chlorite, magnesium chlorite, lithium chlorite, sodium chlorite, and potassium chlorite. A preferred chlorite is sodium chlorite. Additional whitening actives may be hypochlorite and chlorine dioxide. A preferred percarbonate is sodium percarbonate. Other suitable whitening agents include potassium, ammonium, sodium and lithium persjulfates and perborate mono- and tetrahydrates, and sodium pyrophosphate peroxyhydrate.

### Other Active Agents

The present invention may optionally include other agents, such as antimicrobial agents. Included among such agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, among others. Triclosan monophosphate is an additional water soluble antimicrobial agent. The quaternary ammonium agents include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis(2-ethyl-hexyl)-5-methyl hexa hydropyrimidine, benzalkonium chloride, benzethonium chloride and methyl benzethonium chloride are exemplary of typical quaternary ammonium antibacterial agents. Other compounds are bis[4-(R-amino)-1-pyridinium] alkanes as disclosed in U.S. Pat. No. 4,206,215, issued Jun. 3, 1980, to Bailey. Other antimicrobials such as copper salts, zinc salts and stannous salts may also be included. Also useful are enzymes, including endoglycosidase, papain, dextranase, mutanase, and mixtures thereof. Such agents are disclosed in U.S. Pat. No. 2,946,725, Jul. 26, 1960, to Norris et al. and in U.S. Pat. No. 4,051,234, Sep. 27, 1977 to Gieske et al. Specific examples of antimicrobial agents include chlorhexidine, triclosan, triclosan monophosphate, and flavor oils such as thymol. Triclosan and other agents of this type are disclosed in Parran, Jr. et al., U.S. Pat. No. 5,015,466, issued May 14, 1991, and U.S. Pat. No. 4,894,220, Jan. 16, 1990 to Nabi et al. These agents, which provide anti-plaque benefits, may be present at levels of from about 0.01% to about 5.0%, by weight of the dentifrice composition.

### Surfactants

The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976. The present composition typically comprises an anionic surfactant at a level of from about 0.025% to about 9%, from about 0.05% to about 5% in some embodiments, and from about 0.1% to about 1% in other embodiments.

Another suitable surfactant is one selected from the group consisting of sarcosinate surfactants, isethionate surfactants and taurate surfactants. Preferred for use herein are alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the compositions of the present invention from about 0.1% to about 2.5%, preferably from about 0.5% to about 2.0% by weight of the total composition.

Cationic surfactants useful in the present invention include derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc. Preferred compounds are the quaternary ammonium fluorides described in U.S. Pat. No. 3,535,421, Oct. 20, 1970, to Briner et al., where said quaternary ammonium fluorides have detergent properties. Certain cationic surfactants can also act as germicides in the compositions disclosed herein. Cationic surfactants such as chlorhexidine, although suitable for use in the current invention, are not preferred due to their capacity to stain the oral cavity's hard tissues. Persons skilled in the art are aware of this possibility and should incorporate cationic surfactants with this limitation in mind.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577 to Polefka et al., issued Jan. 19, 1993. Typical alkyl dimethyl betaines include decyl betaine or 2-(N-decyl-N,N-dimethylammonio) acetate, coco betaine or 2-(N-coc-N, N-dimethyl ammonio) acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. The betaines of choice are preferably the cocoamidopropyl betaine and, more preferably, the lauramidopropyl betaine.

### Thickening Agents

In preparing toothpaste or gels, thickening agents are added to provide a desirable consistency to the composition, to provide desirable active release characteristics upon use, to provide shelf stability, and to provide stability of the composition, etc. Suitable thickening agents include one or a combination of carboxyvinyl polymers, carrageenan, hydroxyethyl cellulose (HEC), natural and synthetic clays (e.g., Veegum and laponite) and water soluble salts of cellulose ethers such as sodium carboxymethylcellulose (CMC) and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture.

Suitable carboxyvinyl polymers useful as thickening or gelling agents include carbomers which are homopolymers of acrylic acid crosslinked with an alkyl ether of pentaerythritol or an alkyl ether of sucrose. Carbomers are commercially available from B.F. Goodrich as the Carbopol® series, including Carbopol 934, 940, 941, 956, and mixtures thereof.

Copolymers of lactide and glycolide monomers, the copolymer having a number average molecular weight in the range of from about 1,000 to about 120,000, are useful for delivery of actives into the periodontal pockets or around the periodontal pockets as a “subgingival gel carrier.” These polymers are described in U.S. Pat. Nos. 5,198,220, and 5,242,910, issued Mar. 30, 1993 and Sep. 7, 1993, respectively both to Damani, and U.S. Pat. No. 4,443,430, issued Apr. 17, 1984 to Mattei.

Thickening agents are typically present in an amount from about 0.1% to about 15%, preferably from about 2% to about 10%, more preferably from about 4% to about 8%, by weight of the total toothpaste or gel composition, can be used. Higher concentrations may be used for chewing gums, lozenges and breath mints, sachets, non-abrasive gels and subgingival gels.

### Humectants

Another optional carrier material of the present compositions is a humectant. The humectant serves to keep toothpaste compositions from hardening upon exposure to air, to give compositions a moist feel to the mouth, and, for particular humectants, to impart desirable sweetness of flavor to toothpaste compositions. The humectant, on a pure humectant basis, generally comprises from about 0% to about 70%, preferably from about 5% to about 25%, by weight of the compositions herein. Suitable humectants for use in compositions of the subject invention include edible polyhydric alcohols such as glycerin, sorbitol, xylitol, butylene glycol, polyethylene glycol, propylene glycol and trimethyl glycine.

### Flavoring and Sweetening Agents

Flavoring agents may also be added to the compositions. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5%, by weight of the composition.

Sweetening agents which can be used include sucrose, glucose, saccharin, sucralose, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate, sucralose and sodium saccharin, and mixtures thereof. A composition preferably contains from about 0.1% to about 10% of these agents, preferably from about 0.1% to about 1%, by weight of the composition.

In addition to flavoring and sweetening agents, coolants, salivating agents, warming agents, and numbing agents can be used as optional ingredients in compositions of the present invention. These agents are present in the compositions at a level of from about 0.001% to about 10%, preferably from about 0.1% to about 1%, by weight of the composition.

The coolant can be any of a wide variety of materials. Included among such materials are carboxamides, menthol, ketals, diols, and mixtures thereof. Preferred coolants in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al

Suitable salivating agents of the present invention include Jambu® manufactured by Takasago. Examples of warming agents are capsicum and nicotinate esters, such as benzyl nicotinate. Suitable numbing agents include benzocaine, lidocaine, clove bud oil, and ethanol.

### Miscellaneous Carrier Materials

Water employed in the preparation of commercially suitable oral compositions should preferably be of low ion content and free of organic impurities. Water generally comprises from about 5% to about 70%, and preferably from about 20% to about 50%, by weight of the aqueous compositions herein. These amounts of water include the free water which is added plus that which is introduced with other materials, such as with sorbitol.

The present invention may also include an alkali metal bicarbonate salt, which may serve a number of functions including abrasive, deodorant, buffering and adjusting pH. Alkali metal bicarbonate salts are soluble in water and unless stabilized, tend to release carbon dioxide in an aqueous system. Sodium bicarbonate, also known as baking soda, is a commonly used alkali metal bicarbonate salt. The present composition may contain from about 0.5% to about 30%, preferably from about 0.5% to about 15%, and most preferably from about 0.5% to about 5% of an alkali metal bicarbonate salt.

The pH of the present compositions may be adjusted through the use of buffering agents. Buffering agents, as used herein, refer to agents that can be used to adjust the pH of the compositions to a range of about pH 4.0 to about pH 10.0. Buffering agents include sodium bicarbonate, monosodium phosphate, trisodium phosphate, sodium hydroxide, sodium carbonate, sodium acid pyrophosphate, citric acid, and sodium citrate. Buffering agents are typically included at a level of from about 0.5% to about 10%, by weight of the present compositions.

Poloxamers may be employed in the present compositions. A poloxamer is classified as a nonionic surfactant and may also function as an emulsifying agent, binder, stabilizer, and other related functions. Poloxamers are difunctional block-polymers terminating in primary hydroxyl groups with molecular weights ranging from 1,000 to above 15,000. Poloxamers are sold under the tradename of Pluronics and Pluraflo by BASF. Suitable poloxamers for this invention are Poloxamer 407 and Pluraflo L4370.

Other emulsifying agents that may be used in the present compositions include polymeric emulsifiers such as the Pemulen® series available from B.F. Goodrich, and which are predominantly high molecular weight polyacrylic acid polymers useful as emulsifiers for hydrophobic substances.

Titanium dioxide may also be added to the present composition. Titanium dioxide is a white powder which adds opacity to the compositions. Titanium dioxide generally comprises from about 0.25% to about 5% by weight of the dentifrice compositions.

Other optional agents that may be used in the present compositions include dimethicone copolyols selected from alkyl- and alkoxy-dimethicone copolyols, such as C12 to C20 alkyl dimethicone copolyols and mixtures thereof. Highly preferred is cetyl dimethicone copolyol marketed under the trade name Abil EM90. The dimethicone copolyol is generally present in a level of from about 0.01% to about 25%, preferably from about 0.1% to about 5%, more preferably from about 0.5% to about 1.5% by weight. The dimethicone copolyols aid in providing positive tooth feel benefits.

Another optional component of the present compositions is a dentinal desensitizing agent to control hypersensitivity, such as salts of potassium, calcium, strontium and tin including nitrate, chloride, fluoride, phosphates, pyrophosphate, polyphosphate, citrate, oxalate and sulfate.

### Method of Use

The present invention also relates to methods for cleaning teeth and preventing undesirable oral cavity conditions including caries, microbial infection, plaque, calculus, stain and oral malodor and dental erosion.

The method of use herein comprises contacting a subject's dental enamel surfaces and oral mucosa with the oral compositions according to the present invention. The method of use may be by brushing with a dentifrice, rinsing with a dentifrice slurry or mouthrinse, or chewing a gum product. Other methods include contacting the topical oral gel, mouthspray, or other form with the subject's teeth and oral mucosa. It should be understood that the present invention relates not only to methods for delivering the present compositions to the oral cavity of a human, but also to methods of delivering these compositions to the oral cavity of other animals, e.g., household pets or other domestic animals, or animals kept in captivity.

For example, a method of use may include brushing a dog's teeth with one of the dentifrice compositions. Another example would include the rinsing of a cat's mouth with an oral composition for a sufficient amount of time to see a benefit. Pet care products such as chews and toys may be formulated to contain the present oral compositions. The composition is incorporated into a relatively supple but strong and durable material such as rawhide, ropes made from natural or synthetic fibers, and polymeric articles made from nylon, polyester or thermoplastic polyurethane. As the animal chews, licks or gnaws the product, the incorporated active elements are released into the animal's oral cavity into a salivary medium, comparable to an effective brushing or rinsing.

### EXAMPLES

The following examples further describe and demonstrate embodiments within the scope of the present invention. These examples are given solely for the purpose of illustration and are not to be construed as limitations of the present invention as many variations thereof are possible without departing from the spirit and scope.

### Example I Dentifrice Compositions

Dentifrice compositions according to the present invention (IA-IF) and comparative examples (IG and IH) are shown below with amounts of components in weight %. These compositions are made using conventional methods.

| Ingredient | IA | IB | IC | ID | IE | 1F | IG | IH |
|---|---|---|---|---|---|---|---|---|
| Phytic Acid (20% | 4.000 | 2.000 | 0.100 | | | 10.000 | | |
| Soln) | | | | | | | | |
| Sodium Phytate | | | | 10.000 | 0.500 | | | |
| (20% Soln. | | | | | | | | |
| Zinc Carbonate¹ | 2.000 | 1.000 | 2.000 | | | | | |
| Zinc Oxide | | | | 5.000 | | | | |
| Aurichalcite | | | | | 2.000 | | | |
| Zinc | | | | | | 8.000 | | |
| Pyrophosphate | | | | | | | | |
| Zinc Lactate | | | | | | | 2.500 | |
| Na Polyphosphate | | | | | | | 13.000 | |
| Stannous Fluoride | 0.454 | 0.454 | | | 0.454 | | 0.454 | 0.454 |
| Sodium Fluoride | | | 0.243 | 0.243 | | 0.243 | | |
| Sodium Gluconate | 0.672 | 0.600 | | 0.672 | 0.600 | 0.672 | 0.652 | 2.100 |
| Stannous Chloride | | | | 1.500 | | | | 1.500 |
| Sorbitol Soln | 34.275 | 35.785 | 34.275 | 34.275 | 35.785 | 34.275 | | 37.496 |
| Glycerin | | | | | | | 38.519 | 14.425 |
| Hydroxyethyl | 0.300 | 0.300 | 0.300 | 0.300 | 0.300 | 0.300 | | |
| cellulose | | | | | | | | |
| Na CMC | 1.200 | 1.300 | 1.200 | 1.200 | 1.300 | 1.200 | | 0.600 |
| Carrageenan | 0.500 | 0.500 | 0.500 | 0.500 | 0.500 | 0.500 | 0.600 | |
| Xanthan Gum | | | | | | | 0.350 | 0.700 |
| Polyethylene | | | | | | | 7.000 | |
| Glycol | | | | | | | | |
| Propylene Glycol | | | | | | | 7.000 | |
| Silica Abrasive | 20.000 | 16.000 | 20.000 | 20.000 | 16.000 | 20.000 | 25.000 | 20.000 |
| TiO₂ (Anatase) | 0.525 | 0.525 | 0.525 | 0.525 | 0.525 | 0.525 | | 0.525 |
| SLS (28% Soln.) | 4.000 | 7.500 | 4.000 | 4.000 | 7.500 | 4.000 | 2.500 | 5.000 |
| Na Saccharin | 0.250 | 0.250 | 0.250 | 0.250 | 0.250 | 0.250 | 0.500 | 0.300 |
| Flavor | 0.950 | 0.950 | 0.950 | 0.950 | 0.950 | 0.950 | 0.800 | 1.000 |
| NaOH | 0.006 | 0.122 | 0.006 | 0.006 | 0.122 | 0.006 | | 0.600 |
| Na Phosphate | | | | | | | 1.100 | |
| Tribasic | | | | | | | | |
| Water and Minors, | QS | QS | QS | QS | QS | QS | QS | QS |
| e.g., Color soln. | | | | | | | | |

¹Zinc Carbonate AC supplied by Bruggemann Chemical: Newtown Square, PA, USA

### Example II Efficacy of Compositions

Antimicrobial efficacy of the present compositions is measured using the in vitro Plaque Glycolysis and Regrowth Model (i-PGRM). Effectiveness for control of supragingival calculus is defined by activity in prevention of plaque calcification using the Modified Plaque Growth and Mineralization assay. Effectiveness to prevent staining of formulations that contain ingredients associated with staining such as stannous and copper ions is measured using the in vitro Pellicle Tea Stain Model (i-PTSM). Acceptability of formulation aesthetics, such as reduction in astringency, taste acceptability and in-use experience, is measured in controlled consumer testing.

### Antimicrobial Activity

The zinc ion concentration and bioavailability required for the provision of therapeutic actions may differ for different clinical actions, for example, antiplaque vs. gingivitis. However, it is critical to establish a minimum antimicrobial activity level, since the therapeutic activity of zinc can be compromised below this level. To maintain antimicrobial efficacy, it is important to derive a sufficient concentration of zinc ions from the insoluble zinc compound used in the present compositions. Herein, the minimum efficacy provided by the zinc ion source is defined in terms of effects in producing metabolic inhibition of dental plaque bacterial biofilms, which are responsible for numerous undesirable intraoral conditions. Antimicrobial efficacy is thus defined in terms of a noticeable and significant reduction in in situ plaque metabolism as measured using the in vitro Plaque Glycolysis and Regrowth Model (i-PGRM), developed in the Procter & Gamble laboratories.

The i-PGRM is a technique where plaque is grown from human saliva, and treated. with agents designed to produce various levels of antimicrobial activity. The purpose of this technique is to provide a simple and quick method for determining if compounds have a direct effect on the metabolic pathways that plaque microorganisms utilize for the production of toxins which adversely affect gingival health. In particular, the model focuses on the production of organic acids including lactic, acetic, propionic, and butyric. This method utilizes plaque grown on polished glass rods which have been dipped in saliva overnight, soy broth and sucrose for 6 hours, and saliva again overnight. The plaque mass grown on the glass rods is then treated for 1 minute with a 3:1 water to dentifrice slurry. The mass is then placed in a soy broth/sucrose solution for 6 hours and the pH of the incubation solution is measured at the end of the 6 hours. Thus, there are measures of pre-incubation pH and post incubation pH for both test formulations and controls. This testing is typically done with a number of replicates to minimize experimental variances, and a mean pH is calculated from the replicates. Due to strong reactivity with saccharolytic organisms, compositions containing high levels of bioavailable zinc ions produce significant inhibition of plaque acid generation in the i-PGRM assay. This enables formulation variations to be compared for stability and bioavailability of zinc ions with relative ease.

The i-PGRM score is calculated according to the formula:

$$i \text{-} PGRM Score = 100 \% \times \frac{( Test product mean pH - Non \text{-} Zinc Control mean pH )}{( Positive Control mean pH - Non \text{-} Zinc Control mean pH )}$$

The mean pH values refer to incubation media pH's obtained following treatment and sucrose challenge. The negative or non-Zinc control plaque samples produce large amounts of acid, and hence their pH's are lower than that of plaque samples treated with the positive control. The pH difference between the positive and negative controls would typically be a minimum of about 0.6 pH unit, ideally at least about 1.0 pH unit. The negative or non-Zinc control used is a sodium fluoride toothpaste marketed as Crest® Cavity Protection and the positive control is a formulation containing relatively high levels of stannous shown as comparative example IH above and described in U.S. Pat. No. 5,004,597 to Majeti et al. Such high stannous compositions have been shown to produce significant inhibition of plaque acid generation in the i-PGRM assay. A composition as shown in comparative example IG above containing zinc lactate (a soluble zinc compound) was also tested for comparison with the present compositions containing an insoluble zinc compound.

The effectiveness of a formulation prepared from the combination of an insoluble zinc compound and a phytate will ideally be comparable to the positive control, and hence ideal i-PGRM score should approach 100%. As shown in the results of i-PGRM assay in Table 1 below, the present formulations are more effective in inhibiting plaque acid generation than a formulation containing high levels of stannous or a formulation containing a soluble zinc salt.

TABLE 1
Results of i-PGRM Assay of Formulations

| Formulation | i-PGRM Score |
|---|---|
| Non-Zinc Control (Crest ® Cavity Protection) | 0 |
| High Stannous Positive Control (Example IH) | 100 |
| Soluble Zinc Lactate Composition (Example IG) | 84.85 |
| Example IA (2% Zinc Carbonate + Phytate) | 131.75 |
| Example IB (1% Zinc Carbonate + Phytate) | 127.96 |

### Example III Anti-Erosion Efficacy

In addition to the above mentioned therapeutic and cosmetic benefits, the present compositions comprising insoluble zinc salts and phytate also provide protection against the initiation and progression of dental erosion, as demonstrated in a study using an in vitro erosion cycling model. By dental erosion herein is meant a permanent loss of tooth substance from the surface by the action of chemicals, such as harsh abrasives and acids, as opposed to subsurface demineralization or caries caused by bacterial action. Dental erosion is a condition that does not involve plaque bacteria and is therefore distinct from dental caries, which is a disease caused by acids generated by plaque bacteria. It is believed the present compositions deposit on the tooth surface a barrier film or coating thereby protecting teeth from the action of erosive agents on contact.

Human enamel specimens were subjected to a 5 day erosion-cycling regimen. Following an initial pellicle formation, specimens were subjected to seven (7) treatment sequences per day, one (1) hour apart. The treatment sequences consisted of a dentifrice slurry treatment (1 part dentifrice: 3 parts fresh pooled, human saliva [w:w]), saliva remineralization and an erosive acid challenge. At the conclusion of the cycling phase, specimens were analyzed using transverse microradiography (TMR) software. The mean surface loss is reported for each treatment group as microns of enamel lost.

Enamel specimens were prepared by cutting 3-mm cores from extracted, human teeth using a diamond core drill. The teeth, collected by local surgeons, were stored in 5% thymol at room temperature. Enamel cores were mounted in ¼ inch diameter Lucite rods using dental acrylic (Dura Base, Reliance Mfg. Co.) covering all sides except the surface. Polishing with 600 grit silicon carbide-water slurry is used to remove approximately 50 microns of the outer enamel. Following this, specimens are polished for 90 minutes with gamma alumina (Linde No. 3, AB Gamma Polishing Alumina). Enamel specimens found to have surface imperfections are rejected. Following this preparation, nail polish was applied to approximately ⅔ of the surface, ⅓ on each side leaving the center portion exposed as a treatment window. Specimens were randomly assigned to one of the treatment groups (5 specimens/group).

The evening prior to the treatment phase, each group of specimens was placed into 20 ml of fresh, pooled human saliva to initiate the formation of a pellicle layer on the enamel surfaces. To begin the treatment phase, dentifrice slurries were prepared by mixing 5 grams of dentifrice with 15 grams of fresh, pooled human saliva for a period of not less than 4 or more than 5 minutes prior to use. Fresh slurry was prepared for each treatment. Each treatment cycle consisted of: dentifrice slurry (1 min)→rinse in deionized distilled water (ddiH₂O)→saliva (5 min)→erosion challenge (15 min)→rinse in ddiH₂O. There were 7 treatments per day for a total of five treatment days. Dentifrice treatments consisted of immersing the specimens into the dentifrice slurry for one minute while rotating at 75 rpm. The erosion challenge consisted of soaking each treatment group in 20 ml of Cola (at room temperature). A fresh volume of cola was used for each treatment cycle. Saliva was refreshed 3×/day, during the 1^{st}, 4^{th} and 7^{th} treatment cycles. At any time specimens were not in treatment, they remained in 20 ml of pooled, human saliva (stirred). At night, each group of specimens remained immersed in saliva (stirred at room temperature).

After 5 days of treatment, specimens were rinsed well in ddiH₂O and stored refrigerated in a humid environment until analysis. In order to begin the analysis phase, a layer of nail polish was applied to the entire surface of each specimen to seal the surface and protect the fragile eroded areas. Specimens were cut plano-parallel using a hard tissue sectioning saw. Each section was cut to allow the control and treated portion to be represented for analysis. A thin section (˜100 μm) was removed from each specimen and placed flat on a specially designed holder that fits into a camera mounted to an X-ray generator. These sections were then exposed to CuKα radiation. Radiographs were taken using Kodak SO253 Holographic film. The film was processed using standard black and white film developing methods. Radiographic images were then analyzed using TMR, a computer based image analysis system (Inspektor Research). By comparing the original surface, based on the control (untreated) area, to the post treatment surface, the depth of the eroded area can be measured (μM of mineral lost).

Results of the study are summarized in Table 2 below including (1) erosion depth (μm); (2) mean per treatment group; and (3) standard error of the mean (SEM). The data demonstrate the ability of a composition according to the present invention to protect human enamel against the initiation and progression of dental erosion vs. a control treatment group (Crest® Cavity Protection). The present dentifrice comprising 0.4% phytic acid and 1% basic zinc carbonate provided statistically greater protection from erosion than Crest® Cavity Protection.

TABLE 2
Erosion Depth

| | | E depth | | |
|---|---|---|---|---|
| Treatment | Specimen # | (μm) | mean | SEM |
| Crest ® CP | 1 | 15.4 | | |
| | 2 | 9.7 | | |
| | 3 | 20.6 | | |
| | 4 | 18.7 | | |
| | 5 | 8.8 | 14.64 | 2.36 |
| Example IB | 1 | 9.7 | | |
| | 2* | — | | |
| | 3 | 12.0 | | |
| | 4 | 6.9 | | |
| | 5 | 4.0 | 8.15 | 1.55 |

*Specimen was damaged and no measurement was obtained.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition having antimicrobial, antiplaque, anticalculus, anticaries and mouth deodorizing efficacy comprising in an orally acceptable carrier:
(a) from about 0.01% to about 10% by weight of an essentially water-insoluble zinc compound, and
(b) from about 0.01% to about 10% by weight of a polyphosphorylated inositol compound selected from phytic acid, myo-inositol pentakis(dihydrogen phosphate); myo-inositol tetrakis(dihydrogen phosphate), myo-inositol trikis(dihydrogen phosphate), an alkali metal, alkaline earth metal or ammonium salt thereof, and mixtures thereof.

2. An oral care composition according to claim 1, wherein the essentially water-insoluble zinc compound is selected from zinc carbonate, zinc oxide, zinc silicate, zinc phosphate, zinc pyrophosphate, smithsonite, hydrozincite, aurichalcite and rosasite, and mixtures thereof.

3. An oral care composition according to claim 1, wherein the polyphosphorylated inositol compound is selected from phytic acid and its alkali metal, alkaline earth metal or ammonium salt.

4. An oral care composition according to claim 1 further comprising one or more orally acceptable carrier materials selected from fluoride ion sources, anticalculus agents, antimicrobial agents, teeth whitening agents, desensitizing agents, abrasives, chelating agents, thickening agents, buffering agents, alkali metal bicarbonate salts, surfactants, coloring agents, flavor systems, sweetening agents, stain reducing agents, and mixtures thereof

5. An oral care composition according to claim 1, further comprising a fluoride ion source selected from stannous fluoride, sodium fluoride, potassium fluoride, sodium monofluorophosphate, indium fluoride, amine fluoride and mixtures thereof.

6. An oral care composition according to claim 1, further comprising an anticalculus agent selected from linear polyphosphates having an average chain length of from 2 to 125.

7. An oral care composition according to claim 6, wherein the polyphosphate anticalculus agent has an average chain length of from 2 to 21.

8. An oral care composition according to claim 1, further comprising a chelating agent.

9. An oral care composition according to claim 8 wherein the chelating agent is selected from gluconic acid, citric acid, tartaric acid, alkali metal or ammonium salts thereof, and mixtures thereof.

10. An oral care composition according to claim 1, further comprising an antimicrobial agent selected from stannous ion agent, triclosan, triclosan monophosphate, chlorhexidine, domiphen bromide; cetylpyridinium chloride, copper ion agent, essential oils, and mixtures thereof.

11. A method of preventing and controlling plaque, calculus, caries, periodontal disease and mouth malodor in human and animal subjects comprising administering to the subject's oral cavity a composition comprising in an orally acceptable carrier:
(a) from about 0.01% to about 10% by weight of an essentially water-insoluble zinc compound, and
(b) from about 0.01% to about 10% by weight of a polyphosphorylated inositol compound selected from phytic acid, myo-inositol pentakis(dihydrogen phosphate); myo-inositol tetrakis(dihydrogen phosphate), myo-inositol trikis(dihydrogen phosphate), an alkali metal, alkaline earth metal or ammonium salt thereof, and mixtures thereof.

12. A method of protecting human and animal subjects from initiation and progression of dental erosion comprising administering to the subject's oral cavity a composition comprising in an orally acceptable carrier:
(a) from about 0.01% to about 10% by weight of an essentially water-insoluble zinc compound, and
(b) from about 0.01% to about 10% by weight of a polyphosphorylated inositol compound selected from phytic acid, myo-inositol pentakis(dihydrogen phosphate); myo-inositol tetrakis(dihydrogen phosphate), myo-inositol trikis(dihydrogen phosphate), an alkali metal, alkaline earth metal or ammonium salt thereof, and mixtures thereof.
